(19) United States
(12) Patent Application Publication
(10) Pub. No.: US 2018/0185399 A1
(43) Pub. Date: Jul. 5, 2018

(54) GINSENOSIDE C-K ORAL SOLID PREPARATION AND PREPARATION METHOD THEREOF

(71) Applicants: Zhixiong LIAO, Taizhou, Zhejiang (CN); Chunrong FENG, Taizhou, Zhejiang (CN); Jun DAI, Taizhou, Zhejiang (CN); ZHEJIANG HISUN PHARMACEUTICAL CO., LTD., Taizhou, Zhejiang (CN)

(72) Inventors: Zhixiong LIAO, Taizhou, Zhejiang (CN); Chunrong FENG, Taizhou, Zhejiang (CN); Jun DAI, Taizhou, Zhejiang (CN)

(73) Assignee: ZHEJIANG HISUN PHARMACEUTICAL CO., LTD., Taizhou, Zhejiang (CN)

(21) Appl. No.: 15/741,245

(22) PCT Filed: Jul. 3, 2015
(86) PCT No.: PCT/CN2015/000488

§ 371 Date: Dec. 30, 2017

(51) Int. Cl. A61K 31/704; A61K 47/22; A61K 47/32; A61K 47/38; A61K 47/02; A61K 9/00; A61K 9/20; A61K 9/48

(52) U.S. Cl. CPC A61K 31/704; A61K 47/22; A61K 47/32; A61K 9/48; A61K 47/02; A61K 9/0053; A61K 9/2095; A61K 47/38

(57) ABSTRACT

Disclosed are a ginsenoside C-K oral solid preparation and a preparation method thereof. The ginsenoside C-K oral solid preparation is prepared by melting the ginsenoside C-K and a carrier material, extruding and palletizing the mixture, and then mixing the particles and other excipients. The oral solid preparation improves the solubility, bioavailabilty and compliance of ginsenoside C-K.

### FIELD

The present invention relates to the field of medical technology, specifically to a ginsenoside C-K oral solid preparation and method for preparing the same.

### BACKGROUND

Ginsenoside C-K (20-O-β-D-glucopyranosyl-20-(S)-protopanaxadiol) belongs to a tetracyclic triterpene dammarane-type ginsenoside. According to its saponin parent nucleus structure type, it belongs to a protopanaxadiol-type ginsenoside, which is a major degradation product of natural protopanaxadiol-type ginsenosides such as Rb1, Rb2 and Rc in the human gut. This compound was found in 1972, when soil microorganisms were used to digest protopanaxadiol-type ginsenoside to identify authentic ginsenoside aglycone structure. Later on, this compound was also isolated from Panax notoginseng and the fruit of Panax ginseng, but the content thereof is only about 0.03%.

Although the compound has been found and its structure has been identified, the compound has not been given enough attention. In the early nineties of the last century, ginsenoside C-K returned to the scientists' eyes as the deep research on gut metabolism of natural saponins and the mature of anaerobic culture technology. Subsequently, Ginsenoside C-K was isolated from the gut contents of Wistar rats to which ginsenoside Rb2 was orally administered. Since then, a large number of tests have shown that ginsenoside C-K appearing in the rat intestine is not a product of gastric acid hydrolysis but is obtained through degradation by intestinal microorganisms. Furthermore, some scholars have proposed the specific metabolic pathways by which Rb1, Rb2 and Rc are degraded to C-K, which played a landmark role in the study of the active ingredients of ginsenosides in vivo.

After nearly two decades of research, it is found that ginsenoside C-K is a multi-target, highly active substance that not only shows a good activity in anti-tumor, anti-inflammatory, liver protection and anti-allergy, but also a good regulatory role in the nervous system and immune system. However, the very poor water solubility, little absorption in the intestine and low bioavailability of the compound hindered its clinical application. So far, no new drug of ginsenoside C-K has been reported on the market.

In order to solve the problem of water solubility of ginsenoside C-K, a patent document such as CN200410002110 discloses an anti-tumor ginsenoside C-K injection and a preparation method thereof. However, the injection requires the addition of various solubilizers, stabilizers, isotonicity adjusting agents and the like to achieve the requirements of the dosage form itself; also, the unstable solution of ginsenoside C-K may affect the quality and the use time of the injection; more importantly, the injection dosage form is not suitable for the long-term use for clinically chronic diseases, and patient's compliance is not high.

### SUMMARY

An object of the present disclosure is to provide a ginsenoside C-K oral solid preparation with good dissolution rate, high bioavailability and stable quality, and a preparation method thereof by studies on the physicochemical properties of ginsenoside C-K and a large number of screening tests.

The preparation contains melt-extruded granules made of ginsenoside C-K and a carrier material, and other excipient(s).

The preparation is prepared by melting ginsenoside C-K and a carrier material, extruding and granulating, and then mixing the granules with other excipient(s).

For ginsenoside C-K oral solid preparation of the present disclosure, the carrier material is selected from one or more of polyethylene glycol, copovidone, copolymer of polyethylene glycol/vinyl caprolactam/vinyl acetate, hydroxypropyl methyl cellulose acetate succinate and vitamin E polyethylene glycol succinate.

Preferably, the weight ratio of ginsenoside C-K to the carrier material in the ginsenoside C-K oral solid preparation is 1:0.5 to 15, more preferably 1:1 to 5.

Preferably, in the ginsenoside C-K oral solid preparation, the carrier material consists of two of polyethylene glycol, copovidone, copolymer of polyethylene glycol/vinyl caprolactam/vinyl acetate, hydroxypropyl methyl cellulose acetate succinate and vitamin E polyethylene glycol succinate, more preferably copovidone and vitamin E polyethylene glycol succinate.

More preferably, the weight ratio of ginsenoside C-K to the two carrier materials in the ginsenoside C-K oral solid preparation is 1:0.5 to 10:0.1 to 1, even more preferably 1:1 to 5:0.2 to 0.6, most preferably 1:3:0.4.

In addition, other excipient in the ginsenoside C-K oral solid preparation includes one or more of filler, disintegrant, solubilizer and lubricant.

Preferably, the filler in the ginsenoside C-K oral solid preparation is selected from one or more of microcrystalline cellulose, lactose, starch, pregelatinized starch, mannitol and sucrose.

Preferably, the disintegrant in the ginsenoside C-K oral solid preparation is selected from one or more of sodium carboxymethyl starch, crospovidone, low-substituted hydroxypropyl cellulose and croscarmellose sodium.

Preferably, the lubricant in the ginsenoside C-K oral solid preparation is selected from one or more of magnesium stearate, colloidal silica and talcum powder.

Another object of the present disclosure is to provide a preparation method for the ginsenoside C-K oral solid preparation, comprising the steps of: melting ginsenoside C-K and a carrier material by heating; extruding the melted mixture through a hot extruder to obtain granules; mixing the granules with other excipient to give an oral solid preparation.

Compared with ordinary physical mixtures, the dissolution of the ginsenoside C-K oral solid preparation in simulated gastric fluid (pH 1.2) and intestinal fluid (pH 6.8) is improved significantly. This demonstrates that the present disclosure can greatly improve the dissolution performance of ginsenoside C-K.

The in vivo pharmacokinetic studies have shown that under both fasting and fullness conditions, the preparation of the present disclosure can significantly increase the bioavailability of ginsenoside C-K in vivo compared to a physical mixture, indicating a great clinical value.

Compared with the prior art, the ginsenoside C-K oral solid preparation of the present disclosure has the following advantages:

1) The ginsenosides C-K of the present disclosure is dispersed in the matrix in the glassy state to form amorphous raw materials, so as to obtain a highly dispersed product, which increases the solubility of drug substance; it is made into a dosage form suitable for clinical use, which increases the in vivo bioavailability of the ginsenoside C-K, reducing the dose of clinical use and improving clinical compliance.

2) The present disclosure overcomes the influence of high temperature on the quality of ginsenoside C-K during the hot-melt extrusion by reasonably using the carrier material and adjusting the ratio, and the resulting hot melt extrudate has good transparency and is easy to be shaped.

3) The present disclosure requires fewer types of excipients which are all commonly used, and the resulting preparation has a controllable quality and good stability.

4) The preparation process is simple, easy to operate, and suitable for large-scale production.

### BRIEF DESCRIPTION OF DRAWINGS

FIG. 1 is the X-ray diffraction pattern of the ginsenoside C-K drug substance in Example 1.

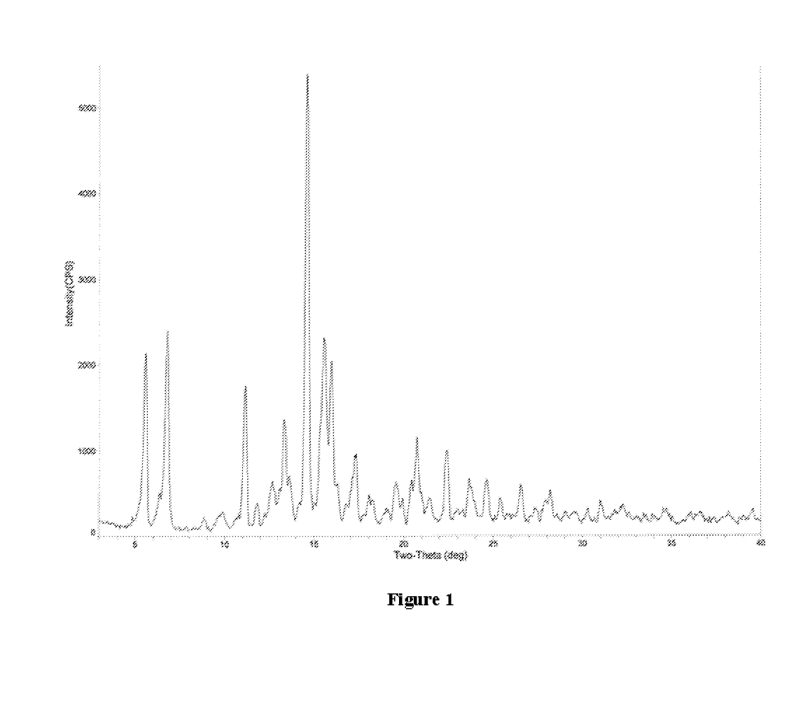

FIG. 2 is the X-ray diffraction pattern of the hot-melt-extrusion granule prepared in Example 1.

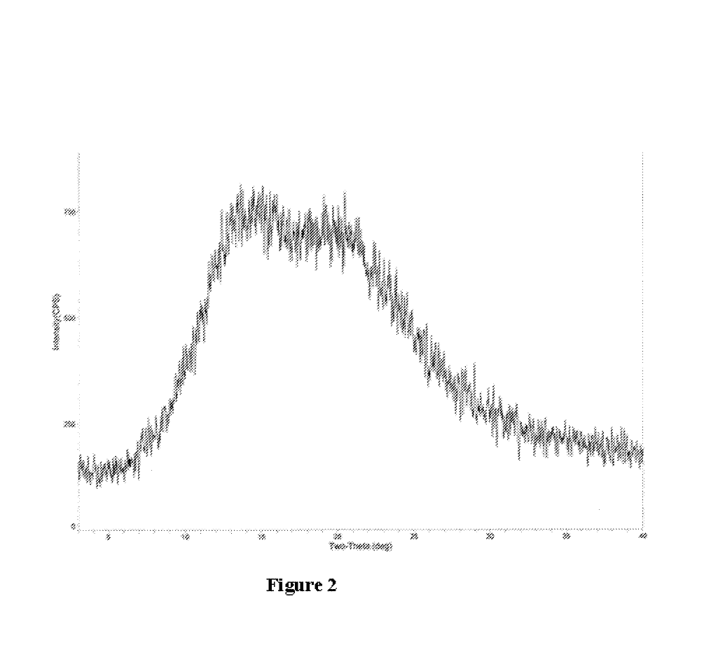

### DETAILED DESCRIPTION

The present disclosure will be described in detail below with reference to the examples. It should be pointed out that the following examples are only used to illustrate the present disclosure, rather than limiting the present invention.

### Example 1

| | | |
|---|---|---|
| Ginsenoside C-K | 1 | part |
| Crospovidone | 3 | parts |
| vitamin E polyethylene glycol succinate (TPGs) | 0.4 | part |
| Microcrystalline cellulose | 5 | parts |
| Crospovidone | 3 | parts |
| Colloidal silicon dioxide | 0.12 | part |
| Magnesium stearate | 0.08 | part |

### Preparation Method:

1) 100 g ginsenoside C-K, 300 g crospovidone and 40 g TPGs were mixed evenly and put into a hot melt extruder preheated to 120˜170° C.; or, 100 g ginsenoside C-K, 300 g crospovidone and 40 g TPGs were simultaneously put into a hot melt extruder preheated to 120˜170° C.;

2) the mixture was extruded, cooled, pulverized and passed through a 20˜80 mesh screen to obtain hot-melt-extrusion ginsenoside C-K granules;

3) 500 g microcrystalline cellulose, 300 g crospovidone and 12 g colloidal silicon dioxide were added to the hot-melt-extrusion ginsenoside C-K granules and mixed evenly, followed by mixed with 8 g magnesium stearate evenly and subjected to compression according to a specification of each tablet containing 50 mg of ginsenoside C-K to obtain tablets.

### Example 2

| | | |
|---|---|---|
| Ginsenoside C-K | 1 | part |
| Copolymer of polyethylene glycol/vinyl | 3 | parts |
| caprolactam/vinyl acetate (Soluplus) | | |
| Microcrystalline cellulose | 4 | parts |
| Lactose | 2 | parts |
| Croscarmellose sodium | 1.5 | parts |
| Colloidal silicon dioxide | 0.12 | part |
| Magnesium stearate | 0.08 | part |

### Preparation Method:

1) 100 g ginsenoside C-K and 300 g Soluplus were mixed evenly and put into a hot melt extruder preheated to 120˜170° C.; or, 100 g ginsenoside C-K and 300 g Soluplus were simultaneously put into a hot melt extruder preheated to 120˜170° C.;

2) the mixture was extruded, cooled, pulverized and passed through a 20˜80 mesh screen to obtain hot-melt-extrusion ginsenoside C-K granules;

3) 400 g microcrystalline cellulose, 200 g lactose, 150 g croscarmellose sodium and 12 g colloidal silicon dioxide were added to the hot-melt-extrusion ginsenoside C-K granules and mixed evenly, followed by mixed with 8 g magnesium stearate evenly, and then pressed into tablets or filled into capsules according to a specification of each tablet or each capsule containing 50 mg of ginsenoside C-K to obtain tablets or capsules.

### Example 3

| | | |
|---|---|---|
| Ginsenoside C-K | 1 | part |
| Crospovidone | 6 | parts |
| vitamin E polyethylene glycol succinate (TPGs) | 0.8 | part |
| Microcrystalline cellulose | 5 | parts |
| Croscarmellose sodium | 3 | parts |
| Colloidal silicon dioxide | 0.12 | part |
| Magnesium stearate | 0.08 | part |

### Preparation Method:

1) 100 g ginsenoside C-K, 600 g crospovidone and 80 g TPGs were mixed evenly and put into a hot melt extruder preheated to 120˜170° C.; or, 100 g ginsenoside C-K, 600 g crospovidone and 80 g TPGs were simultaneously put into a hot melt extruder preheated to 120˜170° C.;

2) the mixture was extruded, cooled, pulverized and passed through a 20˜80 mesh screen to obtain hot-melt-extrusion ginsenoside C-K granules;

3) 500 g microcrystalline cellulose, 300 g croscarmellose sodium and 12 g colloidal silicon dioxide were added to the hot-melt-extrusion ginsenoside C-K granules and mixed evenly, followed by mixed with 8 g magnesium stearate evenly, and then pressed into tablets or filled into capsules according to a specification of each tablet or each capsule containing 50 mg of ginsenoside C-K to obtain tablets or capsules.

### Comparative Example 1

| | | |
|---|---|---|
| Ginsenoside C-K | 1 | part |
| Lactose | 3.18 | parts |
| Microcrystalline cellulose | 1.4 | parts |
| Povidone | 0.3 | part |
| Croscarmellose sodium | 0.12 | part |
| Magnesium stearate | 0.06 | part |

### Preparation Method:

1) 100 g ginsenoside C-K, 318 g lactose, 140 g microcrystalline cellulose, 30 g povidone and 12 g croscarmellose sodium were mixed evenly;

2) 6 g magnesium stearate was added and the mixture was subjected to compression according to a specification of each tablet containing 50 mg of ginsenoside C-K to obtain tablets.

### Example 4 Comparative Dissolution Study

Under fasting conditions, the pH of gastric fluid is about 1.2 and the pH of intestinal fluid is about 6.8. The product of Example 1 and the product of Comparative Example 1 were subjected to comparative dissolution study under the testing conditions as follows:

Dissolution Method: USP II Method (Paddle Method)

Dissolution Medium:

900 ml of dissolution medium at pH 1.2, test samples were collected at 15 min, 30 min, 45 min and 60 min;

900 ml of dissolution medium at pH 6.8, test samples were collected at 15 min, 30 min, 45 min and 60 min.

Rotational speed: 100 rpm

Temperature: 37.5° C.

Test amount: 50 mg (ginsenoside C-K)/cup

Results are shown in the following tables:

### Test Results at pH 1.2 Dissolution Medium

| | |
|---|---|
| pH 1.2 | Dissolution Rate (%) |

| Time | | Comparative |
|---|---|---|
| (min) | Example 1 | Example 1 |
| 0 | 0.00 | 0.00 |
| 15 | 27.27 | 0.44 |
| 30 | 39.76 | 0.28 |
| 45 | 39.74 | 0.17 |
| 60 | 35.96 | 0.00 |

### Test Results at pH 6.8 Dissolution Medium

| | |
|---|---|
| pH 6.8 | Dissolution Rate (%) |

| Time | | Comparative |
|---|---|---|
| (min) | Example 1 | Example 1 |
| 0 | 0.00 | 0.00 |
| 15 | 91.65 | 3.21 |
| 30 | 87.19 | 3.63 |
| 45 | 79.96 | 3.57 |
| 60 | 71.41 | 4.57 |

The dissolution rates of the product of Comparative Example 1 in simulated gastric fluid (pH 1.2) or intestinal fluid (pH6.8) are both less than 10%, while the product of Example 1 has a significantly improved dissolution rate in these two mediums. These results demonstrate that the present disclosure can greatly enhance the dissolution performance of ginsenoside C-K.

### Example 5 Comparative Bioavailability Study

12 male SD rats were divided into 3 groups randomly (4 rats per group) and administered intragastrically the sample of Example 1 and the sample of Comparative Example 1, respectively. 0.25 h, 0.5 h, 1.0 h, 1.5 h, 2.0 h, 3.0 h, 4.0 h, 6.0 h, 8.0 h and 12 h after administration, 0.2 ml venous blood was collected from retro-orbital vein and put into a heparinized tube. Plasma was separated by centrifuge at 11000 rpm for 5 min. The concentration of ginsenoside C-K in the plasma was measured by LC-MS/MS method. Major pharmacokinetic parameters (Tₘₐₓ, Cₘₐₓ, AUC, MRT, T_{1/2}) after rat gastric gavage were calculated by Phoenix 1.3 software. Experimental details are as follows:

Experimental Grouping

| | Animal | Sample | Dosage | Volume | |
|---|---|---|---|---|---|
| Group | number | administered | (mg/kg) | (ml/kg) | Fasting |
| 1 | 4 | Example 1 | 10 | 10 | Yes |
| 2 | 4 | Comparative | 10 | 10 | Yes |
| | | Example 1 | | | |
| 3 | 4 | Example 1 | 10 | 10 | No |
| 4 | 4 | Comparative | 10 | 10 | No |
| | | Example 1 | | | |

Note:
fasting groups were maintained abstinence from food for 12 hours before administration but water ad libitum; and were provided with food 2 hours after administration.
Non-fasting groups had no limitation to foods and water during experiment.

After administration of samples of Example 1 or Comparative Example 1 at a dosage of 10 mg/kg to rats via empty stomach gastric gavage, the ginsenoside C-K concentration in the rat plasma and the pharmacokinetic parameters are shown in the following tables.

### Ginsenoside C-K Concentration in the Plasma (Ng/mL) after Administration of the Samples (10 mg/kg) Via Empty Stomach Gastric Gavage to Rats

| | | Time (h) | | | | | | | | | | |
|---|---|---|---|---|---|---|---|---|---|---|---|---|
| Sample | | 0.25 | 0.5 | 1 | 1.5 | 2 | 3 | 4 | 6 | 8 | 12 | 24 |
| Comparative | Mean | — | 33.1 | 95.1 | 127.5 | 126.0 | 158.5 | 184.1 | 63.5 | 5.8 | 15.4 | — |
| Example 1 | SD | — | 18.5 | 58.4 | 114.4 | 101.7 | 57.5 | 121.3 | 40.1 | 2.5 | 1.1 | — |
| Example 1 | Mean | 21.7 | 26.8 | 42.4 | 162.0 | 149.4 | 192.2 | 181.9 | 158.5 | 32.4 | 4.5 | — |
| | SD | 12.9 | 11.1 | 22.6 | 174.8 | 81.5 | 94.3 | 86.1 | 97.5 | 40.3 | 0.8 | — |

### Pharmacokinetic Parameters after Administration of the Samples (10 mg/kg) Via Empty Stomach Gastric Gavage to Rats

| | | Tₘₐₓ | Cₘₐₓ | AUCₐₗₗ | AUC_{INF}_obs | MRT | T_{1/2} |
|---|---|---|---|---|---|---|---|
| Sample | | (h) | (ng/ml) | (h*ng/ml) | (h*ng/ml) | (h) | (h) |
| Com- | Mean | 3.50 | 222.14 | 766.47 | 985.25 | 3.89 | 1.47 |
| parative | SD | 0.58 | 80.49 | 446.99 | 300.04 | 0.40 | 0.67 |
| Example | CV % | 16.5 | 36.2 | 58.3 | 30.5 | 10.3 | 45.7 |
| 1 | | | | | | | |
| Example | Mean | 4.38 | 274.14 | 1112.23 | 1119.84 | 4.38 | 1.08 |
| 1 | SD | 2.14 | 116.70 | 330.22 | 330.81 | 0.91 | 0.24 |
| | CV % | 48.8 | 42.6 | 29.7 | 29.5 | 20.7 | 21.8 |

After administration of samples of Example 1 or Comparative Example 1 at a dosage of 10 mg/kg to rats via full stomach gastric gavage, the ginsenoside C-K concentration in the rat plasma and the pharmacokinetic parameters are shown in the following tables.

### Ginsenoside C-K Concentration in the Plasma (Ng/mL) after Administration of the Samples (10 mg/kg) Via Full Stomach Gastric Gavage to Rats

| | | Time (h) | | | | | | | | | | |
|---|---|---|---|---|---|---|---|---|---|---|---|---|
| Sample | | 0.25 | 0.5 | 1 | 1.5 | 2 | 3 | 4 | 6 | 8 | 12 | 24 |
| Comparative | Mean | 6.89 | 12.4 | 15.8 | 36.1 | 125.1 | 155.0 | 98.6 | 87.8 | 60.2 | 15.5 | — |
| Example 1 | SD | — | 7.6 | 10.3 | 30.3 | 108.7 | 73.3 | 33.5 | 24.0 | 24.1 | 12.9 | — |
| Example 1 | Mean | 29.1 | 34.5 | 33.1 | 29.1 | 31.1 | 127.9 | 126.5 | 140.3 | 140.8 | 80.3 | 6.16 |
| | SD | 19.7 | 25.5 | 26.6 | 25.4 | 16.9 | 19.9 | 49.9 | 69.5 | 47.0 | 13.7 | — |

### Pharmacokinetic Parameters after Administration of the Samples (10 mg/kg) Via Full Stomach Gastric Gavage to Rats

| | | Tₘₐₓ | Cₘₐₓ | AUCₐₗₗ | AUC_{INF}_obs | MRT | T_{1/2} |
|---|---|---|---|---|---|---|---|
| Sample | | (h) | (ng/ml) | (h*ng/ml) | (h*ng/ml) | (h) | (h) |
| Comparative | Mean | 3.25 | 166.49 | 814.94 | 896.88 | 6.26 | 2.56 |
| Example 1 | SD | 1.89 | 63.69 | 171.45 | 178.62 | 2.26 | 1.79 |
| | CV % | 58.2 | 38.3 | 21.0 | 19.9 | 36.0 | 69.7 |
| Example 1 | Mean | 4.75 | 171.71 | 1367.36 | 2212.69 | 14.48 | 8.13 |
| | SD | 1.50 | 30.25 | 467.23 | 301.57 | 5.61 | 4.21 |
| | CV % | 31.6 | 17.6 | 34.2 | 13.6 | 38.8 | 51.7 |

The in vivo pharmacokinetic studies have shown that consistent pharmacokinetic results were obtained after administration of different ginsenoside C-K samples under both fasting and fullness conditions. For fasting condition, AUC of Example 1 was increased by more than 45% compared to AUC of Comparative Example 1; for fullness condition, AUC of Example 1 was increased by more than 60% compared to AUC of Comparative Example 1, indicating that the present disclosure significantly increased the bioavailability of ginsenoside C-K in vivo.

### Example 5 Stability

The tablets prepared in Example 1 were packed in double aluminum films and placed in the conditions of 40° C.±2° C., RH75%±5% for stability study. The stability data are shown below:

| Index | | 0 month | 1 month | 2 months | 3 months | 6 months |
|---|---|---|---|---|---|---|
| Apparence | | White | White | White | White | White |
| | | tablet | tablet | tablet | tablet | tablet |
| Content | | 102.8% | 104.1% | 103.3% | 102.9% | 103.9% |
| Impurities | Degradation impurity | 0.29% | 0.29% | 0.29% | 0.29% | 0.30% |
| | (RRT 2.4) | | | | | |
| | other largest | 0.17% | 0.17% | 0.17% | 0.17% | 0.16% |
| | single impurity | | | | | |
| | Total impurities | 1.3% | 1.3% | 1.3% | 1.3% | 1.3% |

| | | | | | |
|---|---|---|---|---|---|
| Dissolution rate | 96% | 98% | 94% | 96% | 95% |
| Water content | 4.2% | 4.3% | 3.8% | 4.0% | 3.7% |

The results showed that under the storage conditions of 40° C.±2° C., RH75%±5% for 6 months, no obvious change was detected, demonstrating that the samples are stable.

1. A ginsenoside C-K oral solid preparation, comprising melt-extruded granules made of ginsenoside C-K and a carrier material, and other excipient.

2. The ginsenoside C-K oral solid preparation of claim 1, wherein the preparation is prepared by melting ginsenoside C-K and a carrier material, extruding and granulating, and then mixing the granules with other excipient.

3. The ginsenoside C-K oral solid preparation of claim 1, wherein the carrier material is selected from one or more of polyethylene glycol, copovidone, copolymer of polyethylene glycol/vinyl caprolactam/vinyl acetate, hydroxypropyl methyl cellulose acetate succinate and vitamin E polyethylene glycol succinate.

4. The ginsenoside C-K oral solid preparation of claim 3, wherein the weight ratio of ginsenoside C-K to the carrier material is 1:0.5 to 15.

5. The ginsenoside C-K oral solid preparation of claim 4, wherein the weight ratio of ginsenoside C-K to the carrier material is 1:1 to 5.

6. The ginsenoside C-K oral solid preparation of claim 3, wherein the carrier material consists of two of polyethylene glycol, copovidone, copolymer of polyethylene glycol/vinyl caprolactam/vinyl acetate, hydroxypropyl methyl cellulose acetate succinate and vitamin E polyethylene glycol succinate.

7. The ginsenoside C-K oral solid preparation of claim 6, wherein the carrier material consists of copovidone and vitamin E polyethylene glycol succinate.

8. The ginsenoside C-K oral solid preparation of claim 7, wherein the weight ratio of ginsenoside C-K and copovidone and vitamin E polyethylene glycol succinate is 1:0.5 to 10:0.1 to 1.

9. The ginsenoside C-K oral solid preparation of claim 8, wherein the weight ratio of ginsenoside C-K and copovidone and vitamin E polyethylene glycol succinate is 1:1 to 5:0.2 to 0.6.

10. The ginsenoside C-K oral solid preparation of claim 9, wherein the weight ratio of ginsenoside C-K and copovidone and vitamin E polyethylene glycol succinate is 1:3:0.4.

11. The ginsenoside C-K oral solid preparation of claim 1, wherein the other excipient includes one or more of filler, disintegrant, solubilizer and lubricant.

12. The ginsenoside C-K oral solid preparation of claim 11, wherein the filler is selected from one or more of microcrystalline cellulose, lactose, starch, pregelatinized starch, mannitol and sucrose.

13. The ginsenoside C-K oral solid preparation of claim 11, wherein the disintegrant is selected from one or more of sodium carboxymethyl starch, crospovidone, low-substituted hydroxypropyl cellulose and croscarmellose sodium.

14. The ginsenoside C-K oral solid preparation of claim 11, wherein the lubricant is selected from one or more of magnesium stearate, colloidal silica and talcum powder.

15. A method of preparing the ginsenoside C-K oral solid preparation of claim 1, comprising the steps of: melting the ginsenoside C-K and a carrier material by heating; passing the melted mixture through a hot extruder to obtain granules; mixing the granules with other excipient to give the oral solid preparation.
